(19) United States
(12) Patent Application Publication
(10) Pub. No.: US 2012/0150249 A1
(43) Pub. Date: Jun. 14, 2012

(54) ELECTRICAL CORD FOLDING ENHANCEMENT

(75) Inventors: Frederick Faller, Burlington, MA (US); John Kubat, Townsend, MA (US)

(73) Assignee: ZOLL MEDICAL CORPORATION, Chelmsford, MA (US)

(21) Appl. No.: 12/962,891

(22) Filed: Dec. 8, 2010

(51) Int. Cl. A61N 1/39; H01B 7/00; H02G 15/00

(52) U.S. Cl. 607/5; 174/70 R; 174/135

(57) ABSTRACT

Enhancements may be implemented with respect to an electrical cord or other cable to facilitate folding, storage and deployment thereof. Local enhancement of select regions may predispose the electrical cord to be folded in accordance with a predictable folding assembly. Local enhancement may be accomplished in various ways including, but not limited to, over-molding, co-extrusion, as well as the application of internal or attachable stiffening members.

### FIELD OF THE TECHNOLOGY

One or more aspects relate generally to electrical cords. More particularly, one or more aspects involve electrical cord enhancements to facilitate folding, storage and deployment thereof.

### BACKGROUND

Proper folding and storage of electrical cords may be important for accessibility, including rapid and reliable deployment of associated equipment.

### SUMMARY

Aspects relate generally to folding enhancements for electrical cords and other cables used in connection with equipment in various industries.

In accordance with one or more embodiments, a medical system may comprise a medical device, and an electrical cable having a first connector at a first end connectable to the medical device, a second connector at a second end, and at least one segment along its length between the first end and the second end enhanced to define a prescribed folding arrangement for the electrical cable.

In some embodiments, the electrical cable is stiffened at the at least one segment. The electrical cable may comprise a cable jacket that is over-molded at the at least one segment. The electrical cable may comprise an internal stiffening member at the at least one segment. In other embodiments, a detachable stiffening member may be included at the at least one segment. In still other embodiments, a heat-shrinkable sleeve may be positioned at the at least one segment. The electrical cable may further comprise at least one non-enhanced segment along its length between the first end and the second end. The system may further include a cord storage unit constructed and arranged to receive the electrical cable in the prescribed folding arrangement. In some embodiments, the medical device may be a defibrillator. In at least one embodiment, the electrical cable may comprise a patient therapy cord. An electrode may be attached to the second connector.

In accordance with one or more embodiments, a cable management system may comprise a cable containment vessel, and an electrical cable removably receivable by the cable containment vessel, the electrical cable comprising at least one stiffened section and at least one flexible section, the stiffened and flexible sections may be predisposed to fold the electrical cable in a predetermined arrangement correlated with at least one dimension of the containment vessel.

In some embodiments, the electrical cable may be constructed and arranged to fold without compromising extension or flexibility of the electrical cable. In at least one embodiment, the predetermined arrangement may define an accordion fold of the electrical cable.

In accordance with one or more embodiments, a method of facilitating cable management may comprise providing an electrical cable having at least one section along its length predisposed to fold the electrical cable in a prescribed folding arrangement, and providing a cord storage vessel compatible with the prescribed electrical cable folding arrangement.

In some embodiments, the at least one section is stiffened with respect to a further section of the electrical cable.

In accordance with one or more embodiments, a cable may comprise a first connector at a first end, a second connector at a second end, and at least one segment along its length between the first end and the second end enhanced to define a prescribed folding arrangement for the cable.

In some embodiments, the cable is stiffened at the at least one segment. The cable may further comprise at least one non-enhanced segment along its length between the first end and the second end. The prescribed folding arrangement may comprise an accordion fold.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

Various aspects of at least one embodiment are discussed below with reference to the accompanying figures. The figures are provided for the purposes of illustration and explanation and are not intended as a definition of the limits of the invention. In the figures:

FIG. 1 presents a schematic of an enhanced electrical cord in accordance with one or more embodiments; and

FIG. 2 presents a schematic of a cable management system in accordance with one or more embodiments.

### DETAILED DESCRIPTION

In accordance with one or more embodiments, electrical cords and other cables may be enhanced to facilitate folding and storage thereof in a manner that is consistent, organized and leaves them ready and properly arranged for rapid, reliable deployment. Replacement of cables associated with various equipment, as well as readiness of an associated device for subsequent use is improved, along with the overall appearance and presentation of the cable to a user. Easy and efficient folding of a cable into a prescribed form may be enabled. The folding prescription may not compromise the flexibility of the cord, nor the extension of the cord to its full length. The prescribed folding arrangement or shape may enable the cord and any associated connectors to be provided in a known relationship. The folding arrangement may correspond to an intended containment device to facilitate cable storage. In this way, a prescribed folding may help to achieve stowage of the cords and cables in designated places, as well as facilitate predictable deployment therefrom substantially without tangling, such as for reliable therapy in an emergency situation.

In accordance with one or more embodiments, a conduit, cord or cable may be associated with equipment in various industries including the electronics, consumer products and medical device sectors. Conduits, cords and cables may vary in size, length, geometry, materials and types depending on adaption for intended use. Conduits, cords and cables may transport signals, liquids, gases such as for pneumatics, or other matter. In at least some embodiments, an electrical cord is disclosed. Various types of electrical cords include coaxial, multicore, ribbon, shielded, single and twisted. In some non-limiting embodiments, a cable may be a line cord or patient therapy cable such as, but not limited to ECG, ETCO2 and SPO2 patient cables, including those commercially available from Zoll Medical Corporation of Chelmsford, Mass., the assignee of the present application. For example, a cord may be a medical cable such as those disclosed in U.S. Pat. No. 7,351,912 to Lund et al. which is hereby incorporated herein by reference in its entirety for all purposes. In other embodiments, an electrical cord may be a power cord.

In accordance with one or more embodiments, an electrical cord may include a first end and a second end. A first connector may be positioned at a first end and a second connector may be positioned at the second end. The connectors may facilitate attachment of the electrical cord to a power source or may be appliance-side connectors to interface equipment which the cord is intended to service, including medical devices or components thereof such as defibrillators electrodes. The electrical cord may generally contain one or more conductors, such as fibers or wires, electrically connecting the first connector to the second connector and providing electrical communication therebetween. The construction of the conductor, such as whether it is solid core or fine stranded may impact the flexibility of the electrical cable. The cord or cable may also include other components commonly known to those in the art such as insulation, fillers, sheaths and spacers. Furthermore, a casing or jacket as commonly known in the art may enclose the wires for protection of both its components as well as a user. Various factors including working voltage, current-carrying capacity and various environmental conditions may impact design of the cable.

In accordance with one or more embodiments, an electrical cord or other cable may include one or more segments which are enhanced to facilitate folding. In some non-limiting embodiments, such segments may discourage a user from bending the cord or cable at the enhanced segments. One or more further segments may be substantially unenhanced. In at least some non-limiting embodiments, one or more enhanced segments may be less flexible than one or more further segments. The nature and number of the enhanced and unenhanced segments may predispose a cord to fold in specific areas, encouraging a user to fold the cord in a predefined arrangement. A prescribed folding arrangement may facilitate storage and deployment of the cord in a predictable manner. In some non-limiting embodiments, the prescribed folding arrangement may involve an accordion fold. A predetermined folding arrangement may also be characterized by circular, triangular, rectangular or other desired geometry. Other arrangements and shapes may be implemented depending on an intended application. In some embodiments, the folding arrangement may correspond to a storage device for the cable, cord or conduit or may correspond to facilitate an intended use thereof. The predetermined folding arrangement may be designed to facilitate location of and access to one or more connectors at ends of the cable. The predetermined folding arrangement may also maintain a cable in an orderly manner so as to facilitate deployment and storage thereof. When not in use, the cord or cable may be folded for storage in a manner that will facilitate proper fit in a designated storage device.

The number, positioning, arrangement and size of the substantially enhanced and substantially unenhanced sections may depend on various factors including a desired predefined folding arrangement, storage limitations, desired flexibility, desired durability, geometry of associated storage devices, geometry of associated equipment serviced by the cord, frequency and environment of cord deployment and various characteristics of the cord including its dimensions and materials. In some non-limiting embodiments, enhanced and unenhanced sections may be alternated along a length of the cable. In certain embodiments, a cord may include one, two, three or more enhanced sections as well as one, two, three or more unenhanced sections.

In accordance with one or more embodiments, one or more segments of the electrical cord or cable may be enhanced through stiffening. In some embodiments, certain sections of the cord may be stiffened to prevent folding in those sections while predisposing the cord to fold in one or more further sections. In accordance with one or more embodiments, local stiffening can be accomplished in various ways known to those in the art.

In at least some embodiments, an effective thickness of the sleeve or jacket may be manipulated in one or more enhanced segments to render those segments substantially stiffened in comparison to one or more unenhanced segments. In some embodiments, for example, the cord may be over-molded at one or more enhanced segments. In other embodiments, localized co-extrusion of excess material on the cord jacket may be performed at the enhanced segments. In still further embodiments, internal stiffening members may be added at enhanced segments. In at least one embodiment, attachable stiffening agents may be implemented at one or more enhanced segments. In still other embodiments, heat-shrinkable sleeves may be applied at the one or more enhanced segments.

In other embodiments, materials or material properties may be manipulated to achieve a desired arrangement of enhanced and unenhanced segments. In some embodiments, a cord jacket may be made of a first formulation at the enhanced segments, and of a second formulation at the unenhanced segments. For example, the enhanced and unenhanced segments may be made of different polymers, elastomers or rubbers. Alternatively, the same material may be used in both the enhanced and unenhanced segments, but a property of the material may be altered between them, such as through compounding. For example, one may vary the composition of a particular material (e.g. polyurethane) in order to change its durometer or shore hardness between the enhanced segments and unenhanced segments. This may allow for the same material (but with varying composition) to be used for the entire cord, thus simplifying manufacture.

One or more further segments may be substantially unenhanced to facilitate creation of a prescribed folding arrangement. Unenhanced or unstiffened segments may allow the cord to remain substantially flexible overall as well as to maintain the overall effective length of the cord. The desired degree of enhancement, such as stiffness, of the enhanced segments is subjective and may vary depending on the intended use of the cord. The ratio of the enhanced and unenhanced segments along a length of the cord may also vary depending on intended use.

In accordance with one or more embodiments, a conduit, cable or cord may include indicia to facilitate its deployment, use or storage. In some embodiments, such indicia may correspond to accompanying instructions directed to a user. Indicia may include coding based on color, symbol, text or other system. In at least one embodiment, indicia may facilitate storage of a cable such that one or more connectors positioned at ends of the cable may be easily located and accessed for use.

In accordance with one or more embodiments, a conduit, cable or cord may include further features to facilitate its use and storage, such as a tether or like structure or system intended to constrain at least a portion of its length or otherwise adjust an effective length of free conduit, cable or cord. For example, in some embodiments a portion of a conduit, cable or cord may be constrained, folded or otherwise stored while a further portion remains extended or otherwise available for manipulation and use.

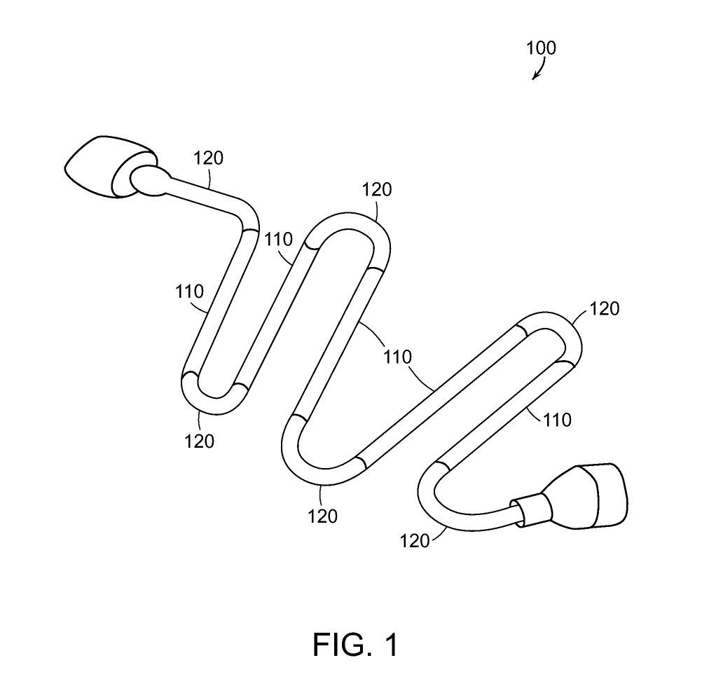

FIG. 1 presents a schematic of an enhanced electrical cord 100 in accordance with one or more embodiments. Cord 100 includes enhanced, stiffened sections 110 and unenhanced, relatively more flexible sections 120. The sections 110, 120 define a prescribed accordion folding arrangement for cord 100.

In accordance with one or more embodiments, a medical system may include a medical device and an electrical cable. The medical system may further include a cable retainer. For example, a medical system may be one such as that disclosed in U.S. Pat. No. 7,530,840 to Lund et al. which is hereby incorporated herein by reference in its entirety for all purposes.

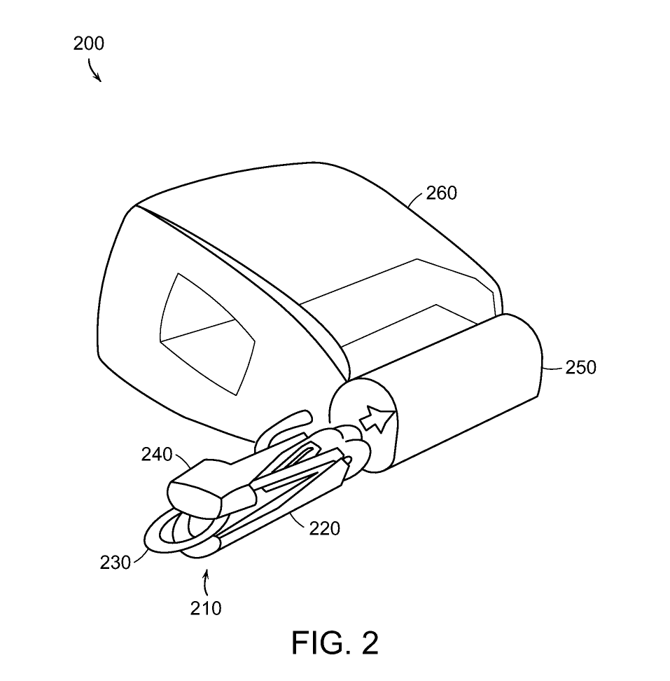

In accordance with one or more embodiments, a cord management system may be provided to facilitate storage of a cord in a known and accessible place. A management system may include an electrical cord and a cord storage unit or containment vessel. The cord storage unit may be constructed and arranged to receive the cord in a prescribed folding arrangement. The electrical cord may be removably receivable by the cable containment vessel. The predetermined folding arrangement may be correlated with at least one dimension of the containment vessel. For example, FIG. 2 presents a schematic of a cable management system 200 comprising electrical cable 210 and cable retainer 250. Cable 210 includes enhanced segments 220 and unenhanced segments 230. Cable 210 folds in a prescribed manner dictated by the positioning and characteristics of segments 220 and 230. The prescribed folding arrangement substantially corresponds to the geometry of retainer 250 to facilitate storage and ease subsequent deployment. The predetermined folding arrangement also facilitates access to connector 240 of cable 210.

Cable management may be facilitated by providing an electrical cable having at least one section along its length predisposed to fold the electrical cable in a prescribed folding arrangement and providing a cord storage vessel compatible with the prescribed electrical cable folding arrangement.

An electrical cord may be retrofitted in accordance with one or more embodiments to facilitate folding, storage and deployment thereof. For example, enhancing features, such as stiffening members, may be applied to one or more segments while one or more further segments may remain substantially unenhanced to promote folding in a prescribed manner.

Having now described some illustrative embodiments, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

It is to be appreciated that embodiments of the devices, systems and methods discussed herein are not limited in application to the details of construction and the arrangement of components set forth in the following description or illustrated in the accompanying drawings. The devices, systems and methods are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A medical system, comprising:
a medical device; and
an electrical cable having a first connector at a first end connectable to the medical device, a second connector at a second end, and at least one segment along its length between the first end and the second end enhanced to define a prescribed folding arrangement for the electrical cable.

2. The system of claim 1, wherein the electrical cable is stiffened at the at least one segment.

3. The system of claim 2, wherein the electrical cable comprises a cable jacket that is over-molded at the at least one segment.

4. The system of claim 2, wherein the electrical cable comprises an internal stiffening member at the at least one segment.

5. The system of claim 2, further comprising a detachable stiffening member at the at least one segment.

6. The system of claim 2, further comprising a heat-shrinkable sleeve positioned at the at least one segment.

7. The system of claim 2, wherein the electrical cable further comprises at least one non-enhanced segment along its length between the first end and the second end.

8. The system of claim 1, further comprising a cord storage unit constructed and arranged to receive the electrical cable in the prescribed folding arrangement.

9. The system of claim 1, wherein the medical device comprises a defibrillator.

10. The system of claim 1, wherein the electrical cable comprises a patient therapy cord.

11. The system of claim 1, wherein an electrode is attached to the second connector.

12. A cable management system, comprising:
a cable containment vessel; and
an electrical cable removably receivable by the cable containment vessel, the electrical cable comprising at least one stiffened section and at least one flexible section, the stiffened and flexible sections predisposed to fold the electrical cable in a predetermined arrangement correlated with at least one dimension of the containment vessel.

13. The system of claim 12, wherein the electrical cable is constructed and arranged to fold without compromising extension or flexibility of the electrical cable.

14. The system of claim 12, wherein the predetermined arrangement defines an accordion fold of the electrical cable.

15. A method of facilitating cable management, comprising:
providing an electrical cable having at least one section along its length predisposed to fold the electrical cable in a prescribed folding arrangement; and
providing a cord storage vessel compatible with the prescribed electrical cable folding arrangement.

16. The method of claim 15, wherein the at least one section is stiffened with respect to a further section of the electrical cable.

17. A cable, comprising:
a first connector at a first end;
a second connector at a second end; and
at least one segment along its length between the first end and the second end enhanced to define a prescribed folding arrangement for the cable.

18. The cable of claim 17, wherein the cable is stiffened at the at least one segment.

19. The cable of claim 17, wherein the cable further comprises at least one non-enhanced segment along its length between the first end and the second end.

20. The cable of claim 17, wherein the prescribed folding arrangement comprises an accordion fold.
